(19) United States
(12) Patent Application Publication
(10) Pub. No.: US 2026/0041431 A1
(43) Pub. Date: Feb. 12, 2026

(54) SYSTEMS AND METHODS FOR MOVING AN IMPLANT WITH A BRAID AND COIL

(71) Applicant: DePuy Synthes Products, Inc., Raynham, MA (US)

(72) Inventors: DANIEL SOLAUN, Miami, FL (US); Pedro D. Pedroso, Parkland, FL (US); Lacey Gorochow, Miami, FL (US)

(21) Appl. No.: 19/263,988

(22) Filed: Jul. 9, 2025

Related U.S. Application Data
(60) Provisional application No. 63/681,483, filed on Aug. 9, 2024.

(51) Int. Cl. A61B 17/12; A61B 17/00

(52) U.S. Cl. CPC A61B 17/12113; A61B 17/1214; A61B 17/12172; A61B 2017/00867

(57) ABSTRACT

An aneurysm intrasaccular implant is provided having both a braid section and a coil. The braid section is expandable from a collapsed configuration to an expanded configuration. The coil includes a proximal coil segment extending proximally from the braid section and a distal coil segment extending distally from the braid section. The implant further includes an engagement member both movable within and confined within the braid section. The engagement member is attached to one or more of the proximal coil segment or the distal coil segment.

### CROSS-REFERENCE TO RELATED APPLICATIONS

The application claims benefit of priority to prior filed U.S. Provisional Patent Application No. 63/681,483 filed Aug. 9, 2024 (Attorney Docket No.: DSP6428USPSP1-243382.000579), which is hereby incorporated by reference in full herein.

### FIELD OF INVENTION

The present disclosure generally relates to medical instruments, and more particularly, to embolic implants for aneurysm therapy.

### BACKGROUND

Cranial aneurysms can be complicated and difficult to treat due to their proximity to critical brain tissues. Prior solutions have included endovascular treatment whereby an internal volume of the aneurysm sac is removed or excluded from arterial blood pressure and flow. Current alternatives to endovascular or other surgical approaches can include intravascularly delivered treatment devices that fill the sac of the aneurysm with embolic material and/or block the entrance or neck of the aneurysm. Such approaches attempt to prevent blood flow into the aneurysm and promote formation of a thrombotic mass within the aneurysm.

Intravascularly delivered aneurysm treatment devices can typically be anchored in place within the aneurysm sac, within the blood vessel, or both. Intrasaccular aneurysm treatment devices, such as embolic coils, are anchored in place primarily within the aneurysm sac.

Intrasaccular aneurysm treatment devices can typically include embolic material to promote formation of thrombotic mass within the aneurysm. Care must be taken when placing the intrasaccular devices so that embolic material and/or a clot formed thereon does not impede the flow of blood in the adjoining parent blood vessel, which can occur if the entrance to the aneurysm is overpacked. Conversely, if the entrance and/or sac is insufficiently packed, blood flow can persist into the aneurysm. Certain aneurysm morphology (e.g., wide neck, bifurcation, etc.) can be particularly challenging to treat, and current treatments of such morphology commonly rely on ancillary devices such as stents or flow diverters positioned within the adjoining blood vessel to cover the aneurysm entrance, protecting the adjoining blood vessel from becoming obstructed and retaining the intrasaccular device within the aneurysm. Placement of ancillary devices can be non-ideal as it can generally increase treatment time and complexity of the treatment procedure compared to treatments that can be performed without ancillary devices. In addition, ancillary devices positioned within the adjoining blood vessel can require antiplatelet treatment for the patient for an extended period of time ranging from months to the rest of their lives.

There is therefore a need for improved methods, implants, and systems for aneurysm treatment.

### SUMMARY

It is an object of the present disclosure to provide systems, implants, and methods to meet the above-stated needs. Generally, it is an object of the present disclosure to provide an implant including a braid section, a proximal coil segment, a distal coil segment, and an engagement member. The braid section is expandable from a collapsed configuration to an expanded configuration. The proximal coil segment extends proximally from the braid section. The distal coil segment extends distally from the braid section. The engagement member is both movable within and confined within the braid section and is attached to one or more of the proximal coil segment or the distal coil segment.

It is also an object of the present disclosure to provide an implant including a braid section, a coil member, and an engagement member. The braid section includes an open proximal end and a distal end. The coil member includes a proximal segment extending through, and movable independently of, the proximal end of the braid section and a distal segment. The engagement member is attached to the proximal segment and positioned within the braid section between the proximal end and the distal end. The distal end of the braid section is configured such that the engagement member cannot pass therethrough.

It is also an object of the present disclosure to provide a method including: advancing a distal coil segment though a microcatheter and into an aneurysm, a braid section and a proximal coil segment trailing the distal coil segment through the microcatheter; advancing the braid section into the aneurysm by pushing a distal end of the braid section with an engagement member attached to the proximal coil segment, the engagement member being movable within the braid section; and advancing the proximal coil segment into the braid section after the braid section expands within the aneurysm, wherein at least a portion of the proximal coil segment can translate through a proximal end of the braid section, wherein the distal end of the braid section is configured such that the engagement member is retained within the braid section proximal to the distal end.

It is also an object of the present disclosure to provide an implant including a braid section and a coil member. The braid section is expandable from a collapsed configuration to an expanded configuration and includes a proximal end and a distal end. The coil member is expandable with the braid section from the collapsed configuration to the expanded configuration and includes a proximal end and a distal end. In this example, either (i) the proximal end of the coil member is attached to the proximal end of the braid section and the distal end of the coil member is movable independently of the distal end of the braid section or (ii) the distal end of the coil member is attached to the distal end of the braid section and the proximal end of the coil member is movable independently of the proximal end of the braid section. The coil member is confined within the braid section in the expanded configuration.

It is also an object of the present disclosure to provide a method including: advancing a distal end of a braid section though a microcatheter and into an aneurysm by pushing a proximal end of the braid section with a delivery device attached to a proximal end of the braid section; advancing the braid section into the aneurysm; and expanding the braid section and a coil member attached to the braid section from a collapsed configuration to an expanded configuration within the aneurysm, the coil member being confined within the braid section in the expanded configuration.

### BRIEF DESCRIPTION OF THE DRAWINGS

The above and further aspects of this disclosure are further discussed with reference to the following description in conjunction with the accompanying drawings, in which like numerals indicate like structural elements and features in various figures. The drawings are not necessarily to scale, emphasis instead being placed upon illustrating principles of the disclosure. The figures depict one or more implementations of the inventive devices, by way of example only, not by way of limitation.

FIG. 1A is a side view of an implant having an engagement member attached to a singular coil member, according to aspects of the present disclosure;

FIG. 1B is a side view of an implant having an engagement member attached to a proximal coil segment that is independent of a distal coil segment, according to aspects of the present disclosure;

FIG. 1C shows the implant from FIG. 1A and details the distalmost portion of the coil member coiled as it would be if it were implanted within an aneurysm, according to aspects of the present disclosure;

FIG. 2A is a schematic showing an implant being positioned at an aneurysm, according to aspects of the present disclosure;

FIG. 2B is a schematic showing a distal coil segment being advanced into the aneurysm, according to aspects of the present disclosure;

FIG. 2C is a schematic showing a braid section being pushed into the aneurysm from its distal end by an engagement member attached to the proximal coil segment, according to aspects of the present disclosure;

FIG. 2D is a schematic showing the implant within the aneurysm and detached from a delivery member, according to aspects of the present disclosure;

FIG. 2E is schematic showing the implant fully implanted within an aneurysm of a vessel, according to aspects of the present disclosure;

FIG. 3A is a schematic showing the implant being removed from a treatment site, the implant having an engagement member attached to a proximal coil segment that is independent of a distal coil segment, according to aspects of the present disclosure;

FIG. 3B is a schematic showing the implant being removed from a treatment site, the implant having an engagement member attached to a singular coil section, according to aspects of the present disclosure;

FIG. 4 is a flowchart showing an example method of advancing an implant into an aneurysm, according to aspects of the present disclosure;

FIG. 5A is a schematic showing an alternative implant disposed in a microcatheter, according to aspects of the present disclosure;

FIG. 5B is a schematic showing the alternative implant of FIG. 5A fully implanted within an aneurysm of a vessel, according to aspects of the present disclosure;

FIG. 6 is a schematic showing a detailed view of an alternate configuration of a terminal end of the alternative implant, according to aspects of the present disclosure;

FIG. 7A is a schematic showing yet another alternative implant disposed in a microcatheter, according to aspects of the present disclosure;

FIG. 7B is a schematic showing the alternative implant of FIG. 7A fully implanted within an aneurysm of a vessel, according to aspects of the present disclosure; and

FIG. 8 is a flowchart showing another example method of advancing an implant into an aneurysm, according to aspects of the present disclosure.

### DETAILED DESCRIPTION

The following detailed description should be read with reference to the drawings, in which like elements in different drawings are identically numbered. The drawings, which are not necessarily to scale, depict selected embodiments and are not intended to limit the scope of the disclosed technology. The detailed description illustrates by way of example, not by way of limitation, the principles of the disclosed technology. This description will clearly enable one skilled in the art to make and use the disclosed technology, and describes several embodiments, adaptations, variations, alternatives and uses of the disclosed technology, including what is presently believed to be the best mode of carrying out the disclosed technology.

In known treatments of aneurysms, a coil can be combined with a mesh at a distal end. As the distal coil is released from a microcatheter at a treatment site, the coil anchors the device within the dome of the aneurysm and provides material/support to push the braid towards the neck to plug the aneurysm. This feature is intended to retain the braid at the neck of the aneurysm and minimize compaction over time. As braid configurations change (braid angle, number of wires, diameter of wires), a braid can be designed stiffer (or softer), more (or less) expandable, and consequently, more (or less) difficult to push. There are trade-offs between the best braid for aneurysm treatment and the ability to push it through a microcatheter. Traditionally, intrasaccular braids are pushed from the proximal end. Depending on the braid configuration, this can cause increased delivery forces as the braid compresses in a microcatheter and wants to expand towards the outside wall of the microcatheter. There are further trade-offs with these devices, such as deliverability (difficulty tracking through a microcatheter), positioning (complete neck coverage to minimize blood flow from entering), and long-term efficacy (the device compacts over time within the aneurysm.

Implants presented herein, in some applications, can be effective to inhibit blood flow circulation in the aneurysm. By inhibiting blood flow circulation, the implant can be effective to inhibit blood flow through a rupture in the aneurysm, particularly for ruptures near the distal portion thereby promoting healing of the rupture and inhibiting blood flow through the rupture.

The designs of the present disclosure are intended to treat an aneurysm by covering the neck with a braid or mesh from within the aneurysm sack. Optimizations are made within the design to minimize compaction over time and maximize deliverability and efficacy. To minimize compaction, a coil is combined with the mesh at the distal end. As the distal coil is released from the microcatheter first, it anchors the device within the dome of the aneurysm and provides material/support to push the braid towards the neck to plug it. This feature is intended to retain the braid at the neck of the aneurysm and minimize compaction over time.

The designs of the present disclosure provide solutions to the aforementioned tradeoffs, as the designs facilitate deliverability of the braid by leading it (i.e., pushing it) from the distal side using a proximal member with an engagement bump, rather than pushing the proximal side of the braid. Leading the braid from the distal end causes the braid to elongate rather than compress, thus improving deliverability. This is facilitated by the use of a proximal member (e.g., a proximal coil segment) with an engagement member, which can be a bump or other enlarged section of the proximal coil segment. The proximal coil segment can be either a continuation of the distal coil segment, or it can be a separate component altogether.

An additional advantage of the designs of the present disclosure design is that as the proximal section is pushed into the aneurysm, it slides through the lumen of the braid, thus filling the braid section with additional material, which may facilitate expansion of the braid to maximize its coverage of the aneurysm neck, as well as provide additional flow disruption at the aneurysm neck as a consequence of extra material within the braid.

As used herein, the terms “about” or “approximately” or “generally” indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 110%. In addition, as used herein, the terms “patient,” “host,” “user,” and “subject” refer to any human or animal subject and are not intended to limit the systems or methods to human use, although use of the subject technology in a human patient represents a preferred embodiment. As well, the term “proximal” indicates a location closer to the operator whereas “distal” indicates a location further away from the operator.

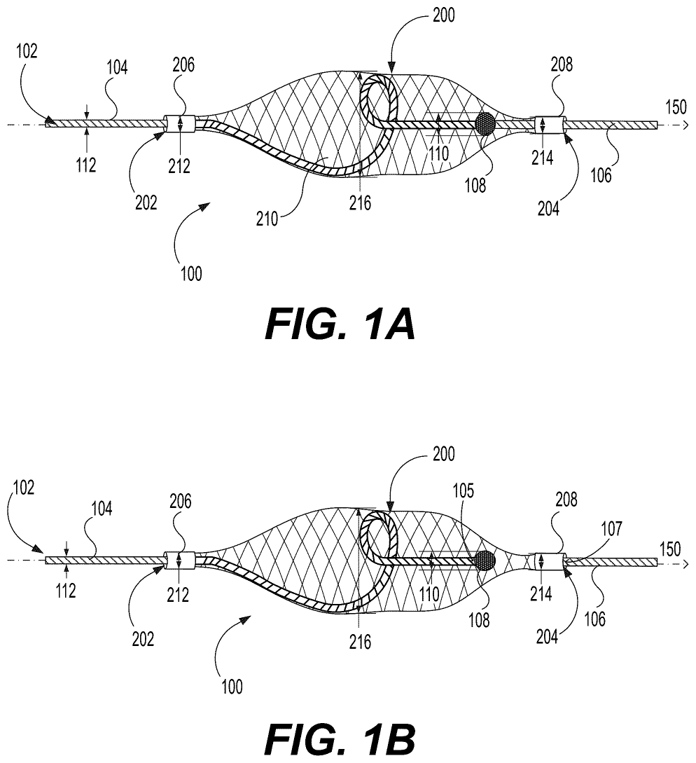

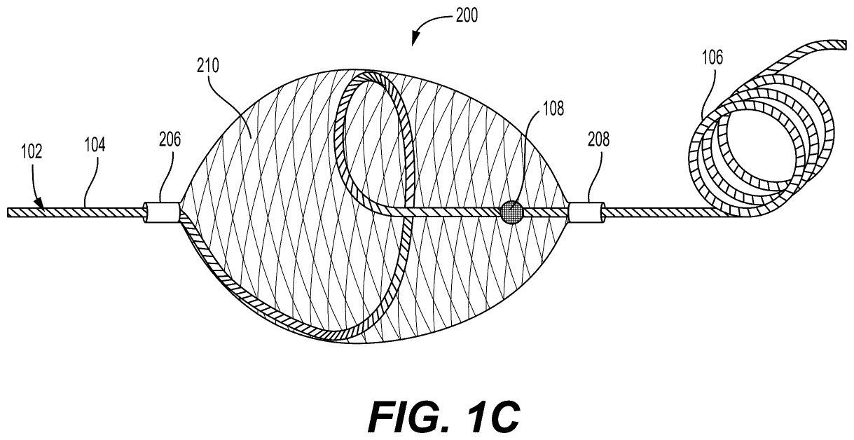

FIG. 1A is a side view of an implant 100 having an engagement member 108 attached to a singular coil member 102, shown in a partially expanded configuration with a portion of a braid section 200 cutaway for the purposes of illustration (it is noted that this cutaway view is also done in the other figures for the same purpose). FIG. 1B is a side view of the implant 100 attached to a proximal coil segment 104 that is independent of a distal coil segment 106, shown in a partially expanded configuration. FIG. 1C shows the implant from FIG. 1A and details the distal most portion of the coil member coiled as it would be if it were implanted within an aneurysm, according to aspects of the present disclosure. FIGS. 1A and 1B illustrate the implant 100 in cross-section, with the implant 100 of FIG. 1B being a variant of the one of FIG. 1A. The implant 100 can treat a range of aneurysm sizes.

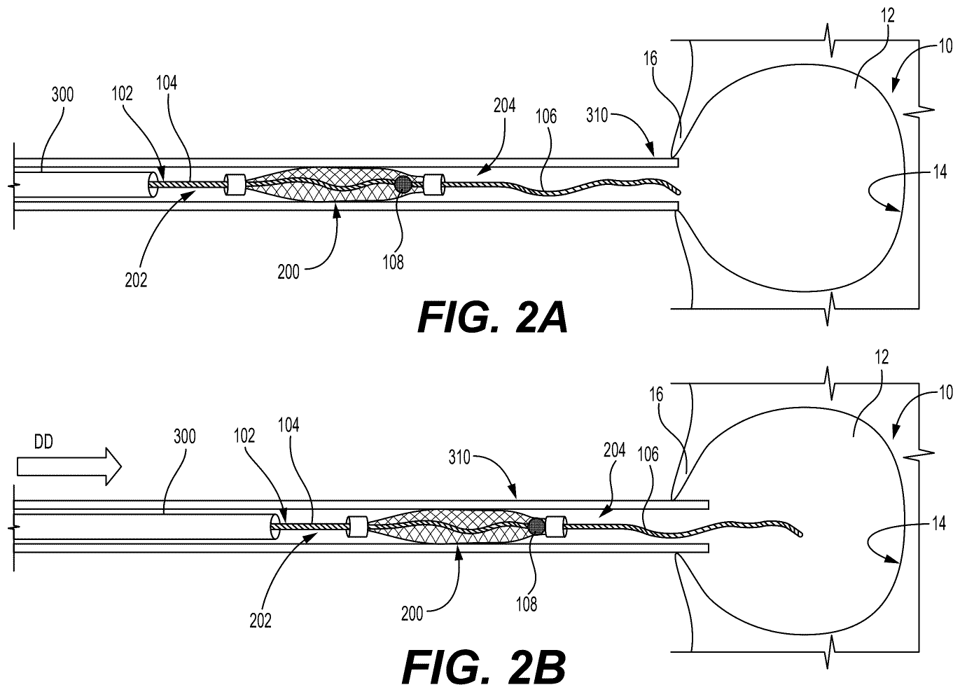

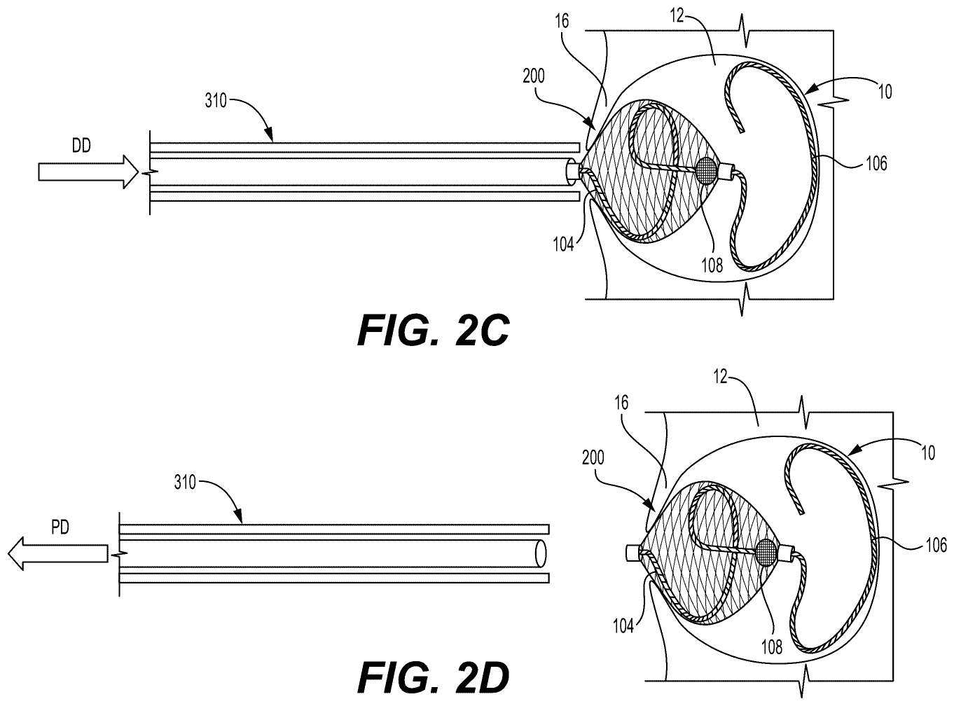

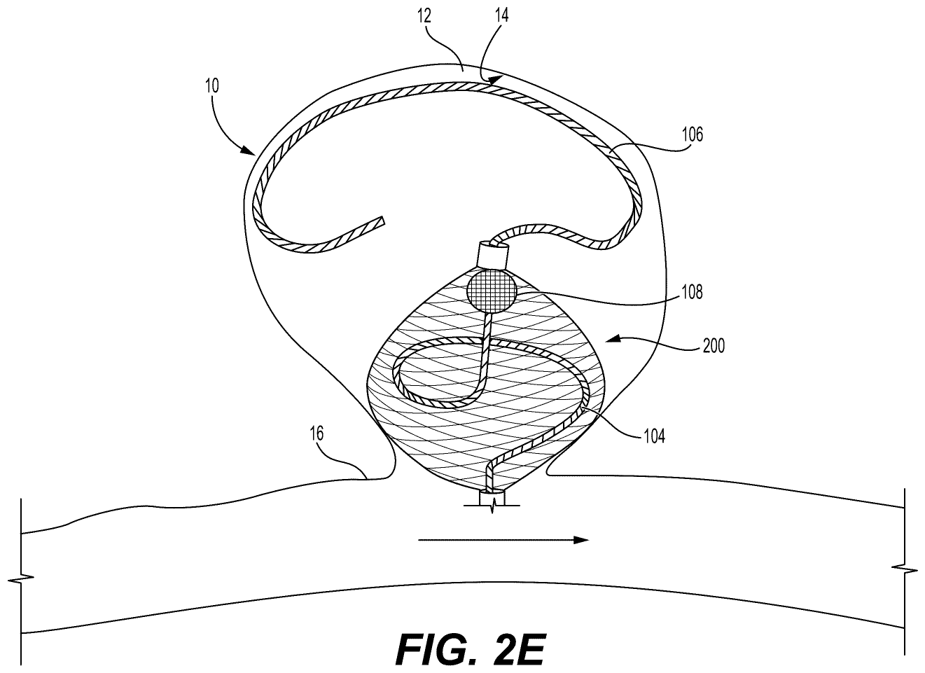

FIGS. 1A-1C are illustrations of an implant 100 having a braid section 200 expandable from a collapsed configuration to an expanded configuration and having a predetermined shape (e.g., as shown in FIG. 1C), a delivery shape (e.g., as shown in FIG. 2A), and one or more implanted shapes (e.g., as shown in FIGS. 2D-2E). In some examples, they can be similar to corresponding shapes disclosed in U.S. patent application Ser. No. 16/418,199 incorporated herein by reference. The braid section 200 can include a mesh shape memory material that is set into the expanded configuration (i.e., the predetermined shape is the expanded configuration). In this configuration, a diameter 214 of the distal end 204 of the braid section 200 is smaller than an intermediate diameter 216 of the braid section 200.

The braid section 200 has an open proximal end 202 and a distal end 204. In the example shown in FIG. 1A, the distal end 204 is also open to allow a distal coil segment 106 (discussed in greater detail below) to pass therethrough. In the example shown in FIG. 1B, the distal end 204 can be open or closed. In some examples, the proximal end 202 of the braid section can include a proximal collar 206, and the distal end 204 of the braid section 200 can include a distal collar 208.

The implant 100 further includes a coil member 102 that includes a proximal coil segment 104 extending proximally from the braid section 200, a distal coil segment 106 extending distally from the braid section 200. The proximal segment 104 extends through and is movable independently of the proximal end 202 of the braid section 200. The segments 104, 106 can be embodied as, e.g., embolic coils. The proximal segment 104 can also be embodied as an implantable wire.

The implant 100 further includes an engagement member 108 attached to coil 102. Engagement member 108 is both movable within and confined within the braid section 200 such that it cannot pass through the proximal end 202 or the distal end 206. In some examples, a diameter 110 of the engagement member 108 is larger than the diameter 214 of the distal end 204 of the braid section 200, which prevents the engagement member 108 from being able to pass through it. As noted above, FIGS. 1A and 1B depict two exemplary configurations of the implant 100, with further differences therebetween discussed below.

In the example shown in FIG. 1A, the engagement member 108 is attached to the proximal segment 104, the distal segment 106, or both segments 104, 106. In this example, the distal segment 106 and the proximal segment 104 are integral and form a single member. More specifically, the distal segment 106 and the proximal segment 104 can form a single length of coil, and the engagement member 108 is attached to the single length of coil (i.e., both the distal segment 106 and the proximal segment). As mentioned above, the implant 100 of FIG. 1A has an open distal end 204, such that the distal segment 106 is movable independently of the distal end 204 of the braid section 200. Similarly, the open proximal end 202 of the braid section 200 permits at least a portion of the proximal segment 104 to slide therethrough.

In the example shown in FIG. 1B, the engagement member 108 is attached to the proximal segment 104 such that movement of the proximal segment 104 causes translation of the engagement member in the braid section 200 while at least a portion of the proximal segment 104 slides through the proximal end 202 of the braid section (discussed in greater detail below). Moreover, the proximal segment 104 is separate from the distal coil segment 106 such that the proximal segment 104 and engagement member 108 are translatable relative to and independently of the distal coil segment 106. A proximal end 107 of the distal segment 106 is attached to the distal end 204 and/or collar 208 of the braid section 200.

In the examples illustrated, and as depicted in the sequence of FIGS. 2A-2E, each braided section 200 can have a delivery shape that is sized to traverse a lumen of a microcatheter 310 to an aneurysm 10 and an implanted shape that is sized to be implanted in the aneurysm sac 12. Each braided section 200 can also have a predetermined shape that is shaped to treat a predetermined range of aneurysm sizes. When the implant 100 exits the catheter 310 and enters the aneurysm 10, the braid 200 and coil 102 can move toward the predetermined shape and be confined by the shape of the aneurysm walls and aneurysm neck 16 such that the resulting implanted shape of the braid 200 is based on the predetermined shape and the anatomy of the aneurysm 10. In some examples, and as depicted, the coil 104 has approximately equal to or greater than the half the diameter of the microcatheter 310 such that the coil 102 only has space to buckle and/or coil inside of the braid section 200 once when it is disposed within the aneurysm 10 (or otherwise in open space), as seen particularly in FIGS. 2C-2D.

In the examples illustrated, each implant 100 can include a locking portion/detachment feature (not shown) that can be engaged to a delivery system (not fully illustrated) including, e.g., a delivery wire 300 to facilitate delivery of the implant through the catheter 310 to the aneurysm 10. The locking portion/detachment feature can be disengaged from the delivery system when the implant is positioned within the aneurysm 10 to allow the delivery system to be extracted. A delivery system suitable for delivering the example implants illustrated herein is disclosed in U.S. patent application Ser. No. 15/964,857 incorporated herein by reference. As would be appreciated and understood by a person of skilled in the art, many other alternative delivery systems can be suitable for delivering example implants disclosed herein.

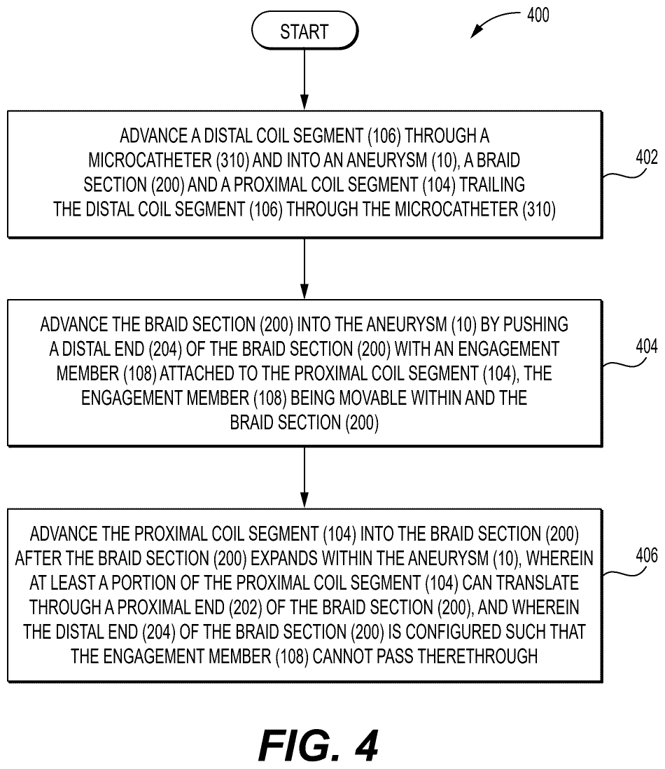

FIG. 4 is a flowchart showing an example method 400 of advancing and/or retracting an implant 100 into/from an aneurysm 10. With reference to FIGS. 2A-2E and 4, the distal coil segment 106 is advanced 402 though a microcatheter 310 in a distal direction DD and into an aneurysm 10, with the braid section 200 and the proximal coil segment 104 trailing the distal coil segment 106 through the microcatheter 310. The braid section 200 is advanced 404 into the aneurysm 10 by pushing the distal end 204 of the braid section 200 with the engagement member 108 attached to the proximal coil segment 104. As seen in FIGS. 2C-2E, the proximal coil segment 104 is advanced at step 406 into the braid section 200 after the braid section 200 expands within the aneurysm 10, where at least a portion of the proximal coil segment 104 can translate through a proximal end 202 of the braid section 200. The proximal coil segment 104 fills in the braid section 200′ to aid in the implant's opening at the neck 16 and to ensure sufficient metal coverage at the neck 16 of the aneurysm 10. The distal segment 106 functions, in part, as a backstop that pushes the braid section 200 towards the neck 16. Further to the above, the distal end 204 of the braid section 200 is configured such that the engagement member 108 is retained within the braid section 200 proximal to the distal end 204. As seen in FIG. 2D, once the implant 100 has been fully inserted into the aneurysm 10, the delivery wire 300 can be detached therefrom and the microcatheter 310 withdrawn in the proximal direction PD.

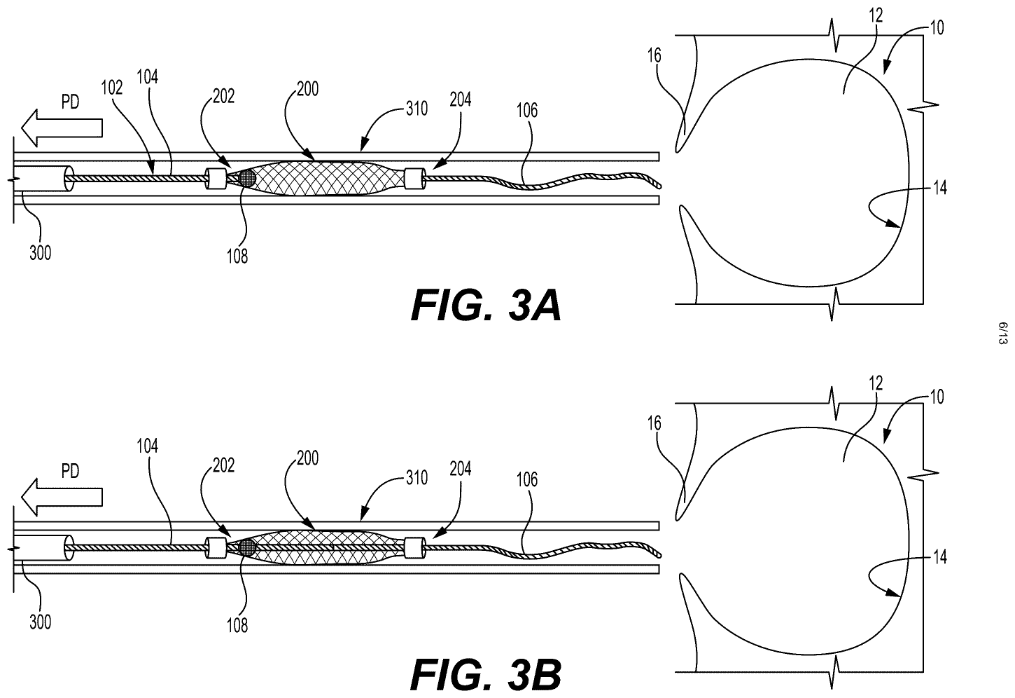

As depicted in FIGS. 3A and 3B, to retract the implant 100 (within the catheter 310 and/or from the aneurysm 10), the proximal segment 104 can be translated in the proximal direction, which translates the engagement member 108 in the proximal direction to engage the proximal end 202 and/or proximal collar 206 to pull the braid section 200 therewith. As seen in FIGS. 3A (which corresponds to the example shown in FIG. 1A) and 3B (which corresponds to the example shown in FIG. 1B), the presently described implant 100 with engagement member 108 is capable of withdrawing the braid section 200 regardless of its attachment (or lack thereof) with the distal segment 106.

Therefore, it will be appreciated that the method 400 can further include (1) pulling the proximal segment 104 proximally in a proximal direction PD such that the engagement member 108 abuts the proximal end 202 of the braid section 200, and (2) retracting the braid section 200 and the distal coil segment 106 from the aneurysm 1) by pulling the braid section 200 proximally via contact of the engagement member 108 with the proximal end 202 of the braid section 200. In some examples, a length of the distal segment 106 can be shorter than the length of the collapsed (elongated) braided section 200, such that the distal segment 106 fully draws into the braid section 200 when the implant is retracted.

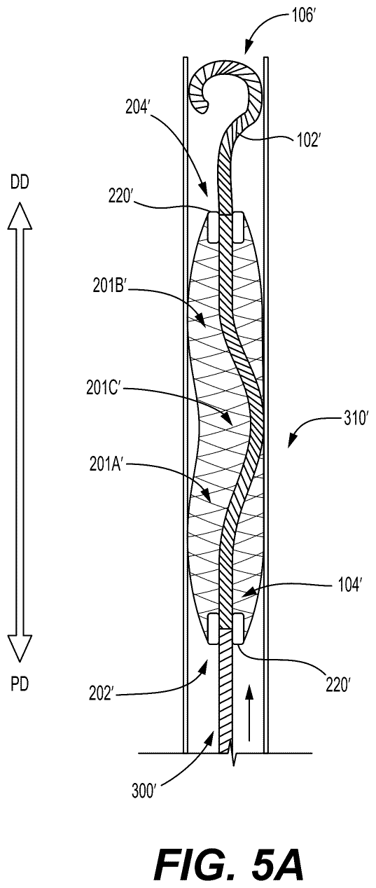

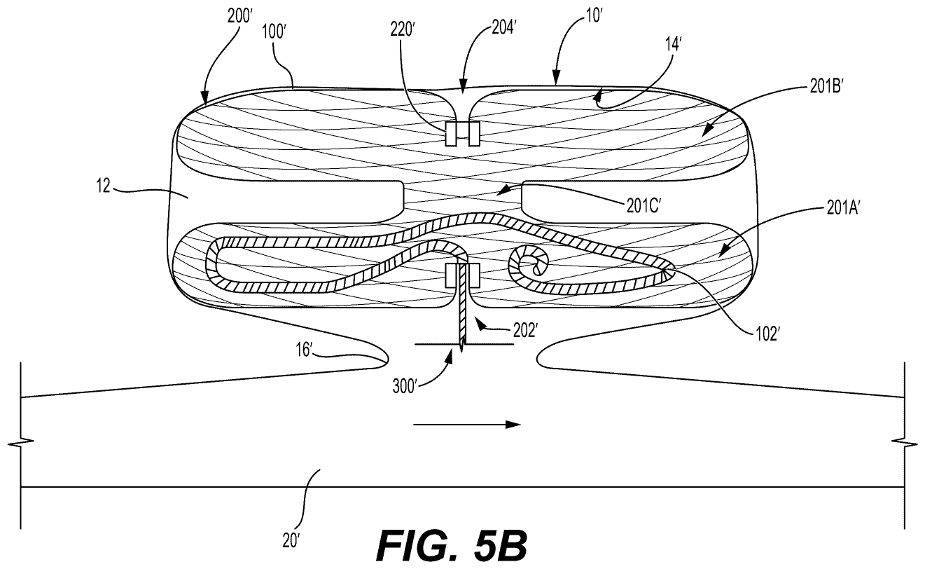

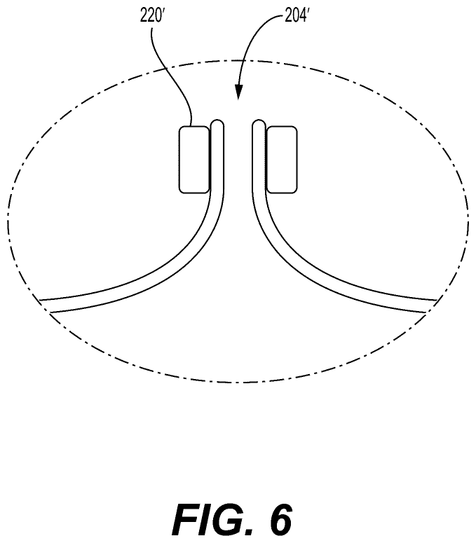

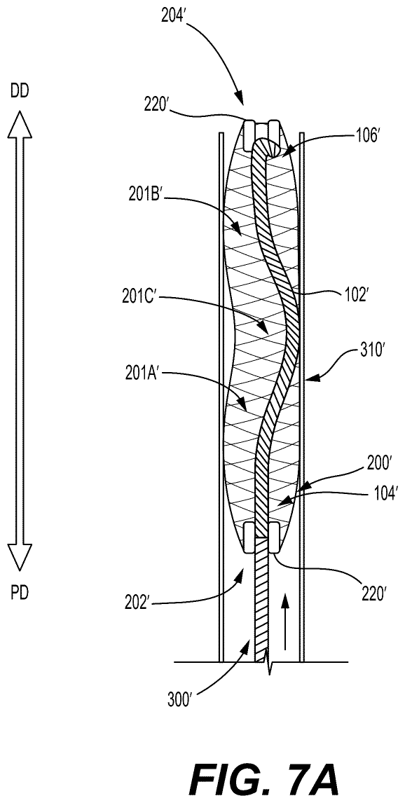

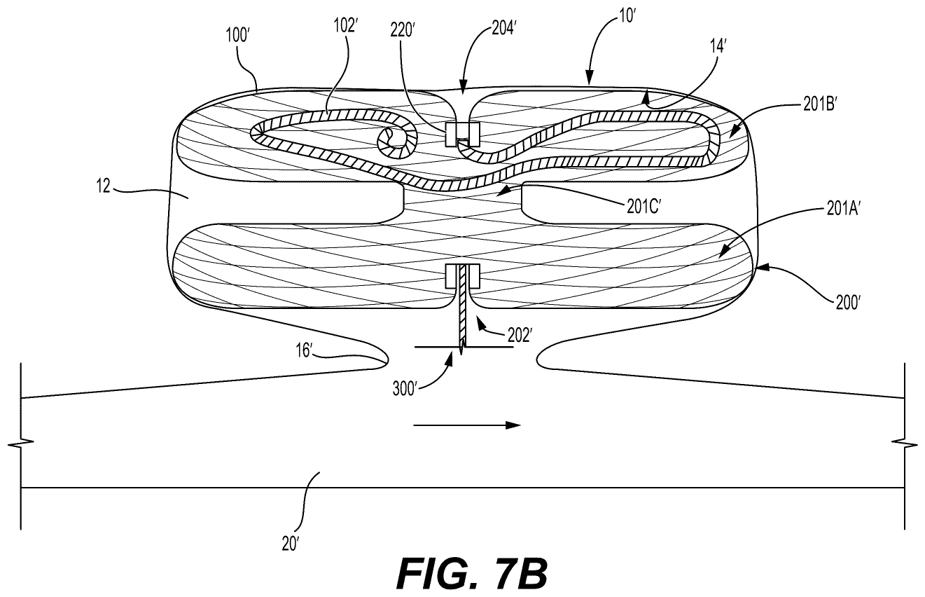

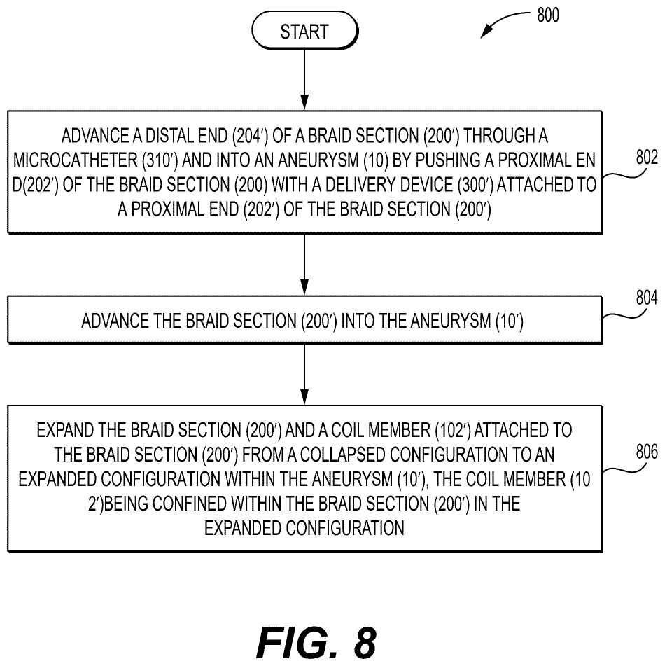

Turning now to FIGS. 5A-8, an alternative design 100′ of the previously described implant 100 is shown. This design 100′ is capable of driving the braid section 200′ without the need for the previously described engagement member 108. FIG. 5A is a schematic showing the alternative implant 100′ disposed in a microcatheter 310. FIG. 5B is a schematic showing the alternative implant 100′ of FIG. 5A fully implanted within an aneurysm of a vessel 20′, according to aspects of the present disclosure. FIG. 6 is a schematic showing a detailed view of an alternative configuration of a terminal end of the alternative implant 100′. FIG. 7A is a schematic showing a modified version of the implant 100′ of FIG. 5A, disposed in a microcatheter 310. FIG. 7B is a schematic showing the alternative implant 100′ of FIG. 7A fully implanted within the aneurysm 10 of a vessel. FIG. 8 is a flowchart showing another example method 800 of advancing an implant into an aneurysm 10.

FIGS. 5A and 5B are illustrations of an implant 100′ having a braid section 200′ expandable from a collapsed configuration to an expanded configuration and having a predetermined shape (e.g., as shown in FIG. 1B), a delivery shape (e.g., as shown in FIG. 2A), and one or more implanted shapes (e.g., as shown in FIG. 5B). In some examples, they can be similar to corresponding shapes disclosed in U.S. patent application Ser. No. 16/418,199 incorporated herein by reference. As discussed in the previous examples, the braid section 200′ can include a mesh shape memory material that is set into the expanded configuration (i.e., the predetermined shape is the expanded configuration).

The braid section 200′ has a proximal end 202′ and a distal end 204′. In the example shown in FIG. 5A, the distal end 204′ is also open to allow a distal coil segment 106 (discussed in greater detail below) to pass therethrough. In some examples, the proximal end 202′ of the braid section can include a proximal marker band 220′ or collar, and the distal end 204′ of the braid section 200′ can include a distal marker band 220′ or collar. As will be appreciated by those skilled in the art, the previously described designs can also employ a marker band in place of or in addition to the aforementioned collars 206, 208. As seen in, for example, FIG. 5A, the marker bands 220′ can extend solely within (i.e., are confined within) the braid section 200′. In other examples, such as shown in FIG. 6, the marker bands 220′ can extend entirely outside of the braid section 200′.

The implant 100′ further includes a coil member 102′ that includes a proximal end 104′ attached to the proximal end 202′ (e.g., the marker band 220′) of the braid section 200′ and a distal end 106′ extending distally from the distal end 204′ of the braid section 200′. The coil member 102′ extends through and is movable independently of the distal end 202′ of the braid section 200′. The coil member 102′ can be embodied as, e.g., an embolic coil. The coil member 102′ is self-expandable from the collapsed configuration to the expanded configuration as the braid section 200′ self-expands (discussed in greater detail below). The coil member 102′ is attached to the proximal end 202′ such that movement of a delivery wire 300′, which is attached to the proximal end 202′ of the braid section 200′ and/or the proximal end 104′ of the coil member 102′, causes translation of the coil member 102′ and the braid section 200′.

In the examples illustrated, and as depicted in FIGS. 5A-5B, each braided section 200′ can have a delivery shape that is sized to traverse a lumen of a microcatheter 310′ (FIG. 5A) to an aneurysm 10′ (FIG. 5B) and an implanted shape that is sized to be implanted in the aneurysm sac 12 (FIG. 5B). Each braided section 200′ can also have a predetermined shape that is shaped to treat a predetermined range of aneurysm sizes. When the implant 100′ exits the catheter 310′ and enters the aneurysm 10′, the braid 200′ can self-expand toward the predetermined shape and be confined by the shape of the aneurysm walls and aneurysm neck 16′ such that the resulting implanted shape of the braid 200′ is based on the predetermined shape and the anatomy of the aneurysm 10′.

As illustrated in FIGS. 5A-5B, the braid 200′ can have a plurality of inversions that define a proximal cavity section 201A′, a distal cavity section 201B′, and an intermediate cavity section 201C′ connecting the proximal and distal cavity sections 201A′, 201B′. As seen best in FIG. 5B, in the expanded configuration, the intermediate cavity section 201C′ has a smaller diameter than a diameter of the proximal cavity section 201A′ and a diameter of the distal cavity section 201B′.

In the collapsed configuration, the distal end 106′ of the coil member 102′ extends out of the braid section 200′ through the distal end 204′ of the braid section 200′. In the expanded configuration, due to the set predetermined shape of the braid section 200′ and the coil member 102′, the distal end 106′ of the coil member 102′ is movable independently of the open distal end 204′ of the braid section 200′ to move therethrough to become confined within the braid section 200′. Thus, in the expanded configuration, both terminal ends 104′, 106′ are confined within the braid section 200′. More specifically, in this configuration, as seen in FIG. 5B, the coil member 102′ is confined approximately entirely or entirely in the proximal cavity section 201A′.

FIGS. 7A-7B depict a variant of the implant 100′ of FIGS. 5A-5B. This implant 100′ is substantially similar to that of the previously described example (FIGS. 5A-B), the difference being the manner in which the coil member 102′ is attached to and moves relative to the braid section 200′. In this example, the distal end 106′ of the coil member 102′ is attached to the distal end 204′ of the braid section 200′ and the proximal end 104′ of the coil member 102′ is movable within the braid section 200′ and independently the proximal end 202′ of the braid section 200′. Moreover, the coil member 102′ is confined within the braid section 200′ in both the collapsed configuration and the expanded configuration (in contrast with the example of FIGS. 5A-5B where a portion of the coil member 102′ extends from the braid section 200′ in the collapsed configuration). In this example, both ends 202′, 204′ of the braid section 200′ can be closed/sealed since the coil member 102′ is confined within and does not move outside of the braid section 200′.

Further to the above, and as seen in FIG. 7A, in the collapsed configuration, the distal end 106′ of the coil member 102′ extends proximal to the proximal end 204′ of the braid section 200′. In the expanded configuration, due to the set predetermined shape of the braid section 200′ and the coil member 102′, the proximal end 104′ of the coil member 102′ is movable independently of the proximal end 202′ of the braid section 200′. Thus, in the expanded configuration, the coil member 102′ self-expands within the braid section 200′. More specifically, in this configuration, as seen in FIG. 7B, the coil member 102′ is confined approximately entirely or entirely in the distal cavity section 201B′.

FIG. 8 is a flowchart showing an example method 800 of advancing the implant 100′ into/from an aneurysm 10′ in accordance with the examples shown in FIGS. 5A-5B and 7A-7B. With reference to 5A and 7A, the distal coil segment 106 is advanced 802 though a microcatheter 310′ in a distal direction DD and into an aneurysm 10′ with a delivery device 300′ (e.g., a delivery wire) attached to a proximal end 202′ of the braid section 200′. The braid section 200′ is advanced 804 into the aneurysm 10′. As seen in FIGS. 5B and 7B, the braid section 200′ and the coil member 102′ attached to the braid section 200′ are expanded 806 from a collapsed configuration to the expanded configuration within the aneurysm 10, the coil member 102′ being confined within the braid section 200′ in the expanded configuration. To retract the implant 100′, the proximal end 202′ can be translated in the proximal direction.

In the examples illustrated, each shape illustrated can be substantially radially symmetrical. Shapes are illustrated in profile unless otherwise stated. Some shapes are illustrated in cross-section. Examples are illustrated with portions of and aneurysm 10, 10′ or a catheter 310, 310′ cut away or in cross-section.

The disclosed technology described herein can be further understood according to the following clauses:
Clause 1. An implant comprising: a braid section expandable from a collapsed configuration to an expanded configuration; a proximal coil segment extending proximally from the braid section; a distal coil segment extending distally from the braid section; and an engagement member both movable within and confined within the braid section, wherein the engagement member is attached to one or more of the proximal coil segment or the distal coil segment.
Clause 2. The implant of Clause 1, wherein the proximal coil segment is separate from the distal coil segment.
Clause 3. The implant of Clause 2, wherein the engagement member is attached to the proximal coil segment, and at least a portion of the proximal coil segment is slidable through a proximal end of the braid section.
Clause 4. The implant of Clause 2, wherein the proximal end of the braid section comprises a proximal collar defining a first diameter of the proximal end, a second diameter of the engagement member being larger than the first diameter of the proximal end.
Clause 5. The implant of Clause 1, wherein a proximal end of the distal coil segment is attached to a distal end of the braid section.
Clause 6. The implant of Clause 5, wherein the engagement member is translatable through the braid section independently of the distal coil segment.
Clause 7. The implant of Clause 5, wherein a distal end of the braid section comprises a distal collar, and wherein the proximal end of the distal coil segment is attached to the distal collar.
Clause 8. The implant of Clause 1, wherein the braid section comprises a shape memory material and is set into the expanded configuration.
Clause 9. An implant comprising: a braid section comprising an open proximal end and a distal end; a coil member comprising: a proximal segment extending through, and movable independently of, the proximal end of the braid section; and a distal segment; and an engagement member attached to the proximal segment and positioned within the braid section between the proximal end and the distal end, wherein the distal end of the braid section is configured such that the engagement member cannot pass therethrough.
Clause 10. The implant of Clause 9, wherein the distal segment and the proximal segment are integral and form a single coil.
Clause 11. The implant of Clause 10, wherein the distal segment and the proximal segment are integral and form a single length of coil, the engagement member being attached to the single length of coil.
Clause 12. The implant of Clause 10, wherein the engagement member is further attached to the distal segment.
Clause 13. The implant of Clause 10, wherein the distal end of the braid section is open and the distal segment extends through, and is movable independently of, the distal end of the braid section.
Clause 14. The implant of Clause 9, wherein a proximal end of the distal segment is attached to the distal end of the braid section.
Clause 15. The implant of Clause 14, wherein the engagement member is translatable through the braid section independently of the distal segment.
Clause 16. The implant of Clause 9, wherein the proximal end of the braid section comprises a proximal collar, and the distal end of the braid section comprises a distal collar.
Clause 17. The implant of Clause 10, wherein the braid section comprises a shape memory material and is set into the expanded configuration, and wherein a first diameter of the distal end of the braid section is smaller than an intermediate dimeter of the braid section, and a second diameter of the engagement member is larger than the first diameter.
Clause 18. A method comprising: advancing a distal coil segment though a microcatheter and into an aneurysm, a braid section and a proximal coil segment trailing the distal coil segment through the microcatheter; advancing the braid section into the aneurysm by pushing a distal end of the braid section with an engagement member attached to the proximal coil segment, the engagement member being movable within the braid section; and advancing the proximal coil segment into the braid section after the braid section expands within the aneurysm, wherein at least a portion of the proximal coil segment can translate through a proximal end of the braid section, wherein the distal end of the braid section is configured such that the engagement member is retained within the braid section proximal to the distal end.
Clause 19. The method of Clause 18, wherein the proximal coil segment is a separate coil segment from the distal coil segment.
Clause 20. The method of Clause 18 further comprising: pulling the proximal coil segment proximally such that the engagement member abuts the proximal end of the braid section; and retract the braid section and the distal coil segment from the aneurysm by pulling the braid section proximally via contact of the engagement member with the proximal end of the braid section.
Clause 21. An implant comprising: a braid section expandable from a collapsed configuration to an expanded configuration and comprising a proximal end and a distal end; a coil member expandable with the braid section from the collapsed configuration to the expanded configuration and comprising a proximal end and a distal end, wherein either (i) the proximal end of the coil member is attached to the proximal end of the braid section and the distal end of the coil member is movable independently of the distal end of the braid section or (ii) the distal end of the coil member is attached to the distal end of the braid section and the proximal end of the coil member is movable independently of the proximal end of the braid section, and the coil member is confined within the braid section in the expanded configuration.
Clause 22. The implant of Clause 21, wherein the braid section and the coil member are configured to self-expand to the expanded configuration.
Clause 23. The implant of Clause 22, wherein the braid section comprises a shape memory material and is set into the expanded configuration.
Clause 24. The implant of Clause 21, further comprising a first marker band connected to one of the proximal end of the braid section or the distal end of the braid section.
Clause 25. The implant of Clause 24, further comprising a second marker band connected to the other of the proximal end of the braid section or the distal end of the braid section.
Clause 26. The implant of Clause 24, wherein the first marker band is disposed solely within the braid section.
Clause 27. The implant of Clause 24, wherein the first marker band is disposed solely outside the braid section.
Clause 28. The implant of Clause 21, wherein the proximal end of the coil member is attached to the proximal end of the braid section and the distal end of the coil member is movable independently of the distal end of the braid section, the distal end of the braid section is open, and the distal end of the coil member extends out of the distal end of the braid section in the collapsed configuration.
Clause 29. The implant of Clause 28, wherein the proximal end of the coil member is confined within the braid section in the collapsed configuration.
Clause 30. The implant of Clause 28, wherein the distal end of the coil member is translatable through the distal end of the braid section.
Clause 31. The implant of Clause 21, wherein the distal end of the coil member is attached to the distal end of the braid section and the proximal end of the coil member is movable independently of the proximal end of the braid section, and the coil member is confined within the braid section in the collapsed configuration.
Clause 32. The implant of Clause 21, wherein the braid section comprises a proximal cavity section, a distal cavity section, and an intermediate cavity section connecting the proximal cavity section and the distal cavity section, and in the expanded configuration, the intermediate cavity section has a smaller diameter than a diameter of the proximal cavity section and a diameter of the distal cavity section.
Clause 33. The implant of Clause 32, wherein, in the expanded configuration, the coil member is confined approximately entirely in the proximal cavity section.
Clause 34. The implant of Clause 32, wherein, in the expanded configuration, the coil member is confined approximately entirely in the distal cavity section.
Clause 35. A method comprising: advancing a distal end of a braid section though a microcatheter and into an aneurysm by pushing a proximal end of the braid section with a delivery device attached to a proximal end of the braid section; advancing the braid section into the aneurysm; and expanding the braid section and a coil member attached to the braid section from a collapsed configuration to an expanded configuration within the aneurysm, the coil member being confined within the braid section in the expanded configuration.

The descriptions contained herein are examples of embodiments of the disclosure and are not intended in any way to limit the scope of the disclosure. As described herein, the disclosure contemplates many variations and modifications of the implants, including alternative braid shapes, alternative coating placements, alternative materials, alternative delivery system engagement/detachment features, alternative braid size, alternative braid porosity, alternative methods for applying a coating to a braid, additional intrasaccular implant structures such as struts or anchors, alternative braid materials, alternative surface enhancement techniques to achieve enhanced cell adhesion to the braid, alternative surface enhancement techniques to achieve enhanced anti-thrombogenic properties of the braid, etc. These modifications would be apparent to those having ordinary skill in the art to which this disclosure relates and are intended to be within the scope of the claims which follow.

What is claimed is:

1. An implant comprising:
a braid section expandable from a collapsed configuration to an expanded configuration;
a proximal coil segment extending proximally from the braid section;
a distal coil segment extending distally from the braid section; and
an engagement member both movable within and confined within the braid section,
wherein the engagement member is attached to one or more of the proximal coil segment or the distal coil segment.

2. The implant of claim 1, wherein the proximal coil segment is separate from the distal coil segment.

3. The implant of claim 2, wherein the engagement member is attached to the proximal coil segment, and at least a portion of the proximal coil segment is slidable through a proximal end of the braid section.

4. The implant of claim 2, wherein the proximal end of the braid section comprises a proximal collar defining a first diameter of the proximal end, a second diameter of the engagement member being larger than the first diameter of the proximal end.

5. The implant of claim 1, wherein a proximal end of the distal coil segment is attached to a distal end of the braid section.

6. The implant of claim 5, wherein the engagement member is translatable through the braid section independently of the distal coil segment.

7. The implant of claim 5, wherein a distal end of the braid section comprises a distal collar, and wherein the proximal end of the distal coil segment is attached to the distal collar.

8. The implant of claim 1, wherein the braid section comprises a shape memory material and is set into the expanded configuration.

9. An implant comprising:
a braid section comprising an open proximal end and a distal end;
a coil member comprising:
a proximal segment extending through, and movable independently of, the proximal end of the braid section; and
a distal segment; and
an engagement member attached to the proximal segment and positioned within the braid section between the proximal end and the distal end, wherein the distal end of the braid section is configured such that the engagement member cannot pass therethrough.

10. The implant of claim 9, wherein the distal segment and the proximal segment are integral and form a single coil.

11. The implant of claim 10, wherein the distal segment and the proximal segment are integral and form a single length of coil, the engagement member being attached to the single length of coil.

12. The implant of claim 10, wherein the engagement member is further attached to the distal segment.

13. The implant of claim 10, wherein the distal end of the braid section is open and the distal segment extends through, and is movable independently of, the distal end of the braid section.

14. The implant of claim 9, wherein a proximal end of the distal segment is attached to the distal end of the braid section.

15. The implant of claim 14, wherein the engagement member is translatable through the braid section independently of the distal segment.

16. The implant of claim 9, wherein the proximal end of the braid section comprises a proximal collar, and the distal end of the braid section comprises a distal collar.

17. The implant of claim 10, wherein the braid section comprises a shape memory material and is set into the expanded configuration, and wherein a first diameter of the distal end of the braid section is smaller than an intermediate dimeter of the braid section, and a second diameter of the engagement member is larger than the first diameter.

18. A method comprising:
advancing a distal coil segment though a microcatheter and into an aneurysm, a braid section and a proximal coil segment trailing the distal coil segment through the microcatheter;
advancing the braid section into the aneurysm by pushing a distal end of the braid section with an engagement member attached to the proximal coil segment, the engagement member being movable within the braid section; and
advancing the proximal coil segment into the braid section after the braid section expands within the aneurysm, wherein at least a portion of the proximal coil segment can translate through a proximal end of the braid section,
wherein the distal end of the braid section is configured such that the engagement member is retained within the braid section proximal to the distal end.

19. The method of claim 18, wherein the proximal coil segment is a separate coil segment from the distal coil segment.

20. The method of claim 18 further comprising:
pulling the proximal coil segment proximally such that the engagement member abuts the proximal end of the braid section; and
retract the braid section and the distal coil segment from the aneurysm by pulling the braid section proximally via contact of the engagement member with the proximal end of the braid section.
